SEQUENCE LISTING (12 sequences)

SEQ ID NO: 1 - DNA, 20 bp, Artificial sequence
  Synthetic sequence
cccacccgcaagcattacca

SEQ ID NO: 2 - DNA, 18 bp, Artificial sequence
  Synthetic sequence
ggttctggccctgctggt

SEQ ID NO: 3 - DNA, 28 bp, Artificial sequence
  Synthetic sequence
tcgctccagagtgaagttcagcaggagc

SEQ ID NO: 4 - DNA, 19 bp, Artificial sequence
  Primer - Synthetic sequence
atcgctgaccgcatgcaga

SEQ ID NO: 5 - DNA, 21 bp, Artificial sequence
  Primer - Synthetic sequence
tcgtcgtactcctgcttggtg

SEQ ID NO: 6 - DNA, 23 bp, Artificial sequence
  Probe - Synthetic sequence
ccatcctggcctcgctgtccacc

SEQ ID NO: 7 - DNA, 19 bp, Artificial sequence
  Primer - Synthetic sequence
cgcaaatcctgatccaacc

SEQ ID NO: 8 - DNA, 19 bp, Artificial sequence
  Primer - Synthetic sequence
actgaagactgcgggattg

SEQ ID NO: 9 - DNA, 25 bp, Artificial sequence
  Probe - Synthetic sequence
agggcctctttaaaagcttgaccga

SEQ ID NO: 10 - DNA, 23 bp, Artificial sequence
  Primer - Synthetic sequence
tgctaaaggtactgacttcgttg

SEQ ID NO: 11 - DNA, 22 bp, Artificial sequence
  Primer - Synthetic sequence
gattctttgcacttatggaacg

SEQ ID NO: 12 - DNA, 22 bp, Artificial sequence
  Probe - Synthetic sequence
atggtgatggtgtcacttggga

(19) United States
(12) Patent Application Publication
(10) Pub. No.: US 2024/0279737 A1
(43) Pub. Date: Aug. 22, 2024

(54) DETERMINATION OF CELLULAR KINETICS AND BIODISTRIBUTION FOR CAR-T CELLS

(71) Applicant: TAKEDA PHARMACEUTICAL COMPANY LIMITED, Osaka (JP)

(72) Inventors: Hiroshi SUGIMOTO, Cambridge, MA (US); Xiaodong FANG, Cambridge, MA (US)

(21) Appl. No.: 18/248,604

(22) PCT Filed: Oct. 12, 2021
(86) PCT No.: PCT/IB2021/000678

§ 371 Date: Apr. 11, 2023

Related U.S. Application Data
(60) Provisional application No. 63/090,479, filed on Oct. 12, 2020.

(51) Int. Cl. C12Q 1/6881; C12Q 1/6806; C12Q 1/6851

(52) U.S. Cl. CPC C12Q 1/6881; C12Q 1/6851; C12Q 1/6806; C12Q 2600/156; C12Q 2600/166

(57) ABSTRACT

Provided herein, among other things, is a method of determining a transgene copy number in a biological sample, the method comprising: (a) extracting genomic DNA (gDNA) from the biological sample using an automated process, thus isolating gDNA; (b) performing digital droplet PCR (ddPCR) on the isolated gDNA using one or more primers to amplify a transgene of interest and to amplify a reference gene, thus normalizing gDNA input; and (c) determining transgene copy number in the biological sample.

### CROSS-REFERENCE TO RELATED APPLICATIONS

This application is a 35 U.S.C. 371 National Stage Application of International Application No. PCT/IB2021/000678, filed on Oct. 12, 2021, which claims priority to U.S. Provisional Application Ser. No. 63/090,479, filed Oct. 12, 2020. The contents of each of the foregoing applications are hereby incorporated by reference in their entireties.

### SEQUENCE LISTING

The instant application is being filed with an electronically filed Sequence Listing in ASCII text format. The sequence listing file entitled MIL_008US1_SL.TXT, was created on Dec. 13, 2023, and is 2,737 bytes in size; the information in electronic format of the Sequence Listing is incorporated herein by reference in its entirety.

### BACKGROUND

Chimeric antigen receptors (CARs) T cells are characterized by genetically engineered receptors that can be programed to both recognize a specific antigen and, when bound to that antigen, activate an immune cell to attack and destroy a cell bearing that antigen. When these antigens exist on tumor cells, an immune cell that expresses the CAR can target and kill the tumor cell.

The unprecedented positive clinical response of chimeric antigen receptor T (CAR-T) cell therapy for CD19-positive aggressive B-cell lymphoma and B-cell precursor acute lymphoblastic leukemia have brought adoptive cell immunotherapy into the mainstream of global hematologic cancer treatment. However, challenges of CAR-T cell therapy still remain. Of these remaining challenges, the characterization of in vivo cellular kinetics and biodistribution of chimeric antigen receptor T (CAR-T) cells is at the forefront. To date, a standardized, reliable assay to characterize cellular distribution, expansion, contraction and persistence profiles does not exist.

### SUMMARY

The present invention provides a method of determining a transgene copy number in a biological sample. The inventors have surprisingly discovered a standardized, reliable assay to assess biodistribution and cellular kinetics of cells of interest. As explained below, in some embodiments, the assay uses digital droplet PCR to assess the presence of a transgene of interest in a biological sample.

In some aspects, a method of determining a transgene copy number in a biological sample is provided, the method comprising: (a) extracting genomic DNA (gDNA) from the biological sample using an automated process, thus isolating gDNA; (b) performing digital droplet PCR (ddPCR) on the isolated gDNA using one or more primers to amplify a transgene of interest and to amplify a reference gene, thus normalizing gDNA input; and (c) determining transgene copy number in the biological sample.

In some embodiments, one reference gene is amplified. In some embodiments, more than one reference gene is amplified. For example, in some embodiments, 1, 2, 3, 4, or 5 reference genes are amplified. In some embodiments, more than 5 reference genes are amplified.

In some embodiments, the transgene copy number in the biological sample is measured in copy number/μg gDNA.

In some embodiments, the transgene copy number is determined using the following formula:

$$\frac{copy}{μg gDNA} = \frac{\frac{transgene copy}{μL reaction} \times 1 , 000 , 000}{\frac{reference gene copy}{μL reaction} \times \frac{3.3 pg}{copy}} .$$

In some embodiments, the copy number/μg gDNA unit is converted to copy number/μL blood by measuring gDNA extraction recovery.

In some embodiments, gDNA extraction recovery is assessed by spiking a known amount of a control gene before and after the gDNA extraction process.

In some embodiments, gDNA extraction recovery is stable.

In some embodiments, the gDNA extraction is stable as assessed by a quality control concentration of a control exogenous gene.

In some embodiments, the gDNA recovery is at least 50%, 55%, 60%, 65%, 70%, 75%, 80%, 85%, 90%, 95%, 99% or 100%. For example, in some embodiments, the gDNA recovery is at least 50%. In some embodiments, the gDNA recovery is about 55%. In some embodiments, the gDNA recovery is about 60%. In some embodiments, the gDNA recovery is about 65%. In some embodiments, the gDNA recovery is about 70%. In some embodiments, the gDNA recovery is about 75%. In some embodiments, the gDNA recovery is about 80%. In some embodiments, the gDNA recovery is about 85%. In some embodiments, the gDNA recovery is about 90%. In some embodiments, the gDNA recovery is about 95%. In some embodiments, the gDNA recovery is about 99%. In some embodiments, the gDNA recovery is about 100%.

In some embodiments, wherein the quality control concentration is selected from low quality control (LQC), medium quality control (MQC), or high quality control (HQC) concentration. Accordingly, in some embodiments, the quality control concentration is low quality control (LQC) concentration. In some embodiments, the quality control concentration is selected from medium quality control (MQC) concentration. In some embodiments, the quality control concentration is selected from high quality control (HQC) concentration.

In some embodiments, gDNA extraction recovery is assessed by spiking a known amount of a chimeric antigen receptor (CAR) T cell before the gDNA extraction process.

In some embodiments, the method does not comprise a sorting step.

In some aspects, a method of determining a transgene copy number in a biological sample is provided, the method comprising: (a) extracting genomic DNA (gDNA) from the biological sample using an automated process, thus isolating gDNA; (b) determining a genomic DNA recovery rate by spiking in a known amount of a control gene before and after the gDNA extraction process; (c) performing digital droplet PCR (ddPCR) on the isolated DNA using one or more primers to amplify a transgene of interest and to amplify the control gene, thus normalizing gDNA input; (d) determining transgene copy number in the biological sample.

In some embodiments, transgene copy number in the biological sample is measured in copy number/μL blood. In some embodiments, transgene copy number in the biological sample is measured by copy number/mg tissue.

In some embodiments, the transgene copy number is determined using the following formula:

$$\frac{copy}{μ L blood} = \frac{transgene copy}{μ L of PCR reaction buffer} \times \frac{total μ L of PCR reaction buffer}{μ L of eluate used} \times \frac{total μ L of eluate}{total μ L of blood used} \times \frac{1}{genomic DNA recovery ( \% )} .$$

In some embodiments, the transgene copy number is in the biological sample is measured in copy number/mg tissue.

In some embodiments, the methods described herein can be applied to all biofluids and tissue samples. In some embodiments, the biomatrix sample is selected from blood, liver, kidney, or tumor homogenate. Accordingly, in some embodiments, the biofluid sample is blood. In some embodiments, the tissue sample is liver. In some embodiments, the tissue sample is kidney. In some embodiments, the tissue sample is tumor homogenate.

In some embodiments, the transgene copy number is determined using the following formula:

$$\frac{copy}{mg tissue} = \frac{transgene copy}{μ L of PCR reaction buffer} \times \frac{total μ L of PCR reaction buffer}{μ L of eluate used} \times \frac{total μ L of eluate}{total mg of tissue used} \times \frac{1}{genomic DNA recovery ( \% )} .$$

In some embodiments, the limit of quantitation (LOQ) is less than 50 copies of the transgene per 1 μg of gDNA. In some embodiments, the LOQ is about between 40-50 copies of the transgene per 1 μg of gDNA. In some embodiments, the LOQ is about between 30 and 50 copies of the transgene per 1 μg of gDNA.

In some embodiments, the reference gene is a single copy gene per haploid human genome. In some embodiments, the reference gene is a single copy gene per haploid mouse genome. In some embodiments, the reference gene is a single copy gene per haploid human genome and mouse genome.

In some embodiments, the reference gene is selected from a single copy gene such as alpha actin, RNase P gene, gamma tubulin 1, or GAPDH. Accordingly, in some embodiments, the reference gene is alpha actin. In some embodiments, the reference gene is RNase P. In some embodiments, the reference gene is gamma tubulin 1. In some embodiments, the reference gene is GAPDH. In some embodiments, any single copy reference gene having a single copy in both the human genome and the mouse genome is suitable for the methods described herein.

In some embodiments, selectivity of ddPCR is assessed.

In some embodiments, the selectivity of ddPCR is assessed by performing a measurement of a no template control sample.

In some aspects, a method of determining cellular kinetics of administered CAR-T cells is provided, the method comprising determining a CAR-T transgene copy number in accordance with the disclosure provided herein.

In some aspects, a method of determining dose selection and dosing schedule for administration of CAR-T cells to a subject in need thereof is provided, the method comprising determining CAR-T transgene copy number in a biological sample in accordance with any of the preceding claims, and adjusting CAR-T dose selection and/or dosing schedule to achieve a therapeutically effective amount of CAR-T cells in the subject.

In some aspects, a kit for determining a transgene copy number in a biological sample is provided, comprising primers to amplify a transgene of interest and to amplify a reference gene, and instructions for determining the transgene copy number in the biological sample.

In some embodiments, the instructions for determining the transgene copy number comprises the following formula:

$$\frac{copy}{μg gDNA} = \frac{\frac{transgene copy}{μL reaction} \times 1,000,000}{\frac{reference gene copy}{μL reaction} \times \frac{3.3 pg}{copy}} .$$

In some aspects, a kit for determining a transgene copy number in a biological sample is provided, comprising primers to amplify a transgene of interest and to amplify a reference gene, one or more purified control genes, and instructions for determining the transgene copy number in the biological sample.

In some embodiments, the instructions for determining the transgene copy number comprises the following formula:

$$\frac{copy}{μ L blood} = \frac{transgene copy}{μ L of PCR reaction buffer} \times \frac{total μ L of PCR reaction buffer}{μ L of eluate used} \times \frac{total μ L of eluate}{total μ L of blood used} \times \frac{1}{genomic DNA recovery ( \% )} .$$

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### BRIEF DESCRIPTION OF THE DRAWINGS

FIG. 1 depicts a method of determining CAR-T cells copy number in a biological sample using a droplet digital polymerase chain reaction (ddPCR).

FIG. 2 depicts a workflow of quantitative cellular kinetics and tissue distribution assay using ddPCR.

FIG. 3 depicts primers and probes for CAR-T cells used in running the ddPCR for determining the cellular kinetics of CAR-T cells.

FIG. 4A depicts optimization of duplexing capability of CAR-T transgene and alpha actin detection by ddPCR. FIG. 4A establishes that 100 nM is the optimum concentration of each primer as at this concentration the positive and negative signals were well separated. FIG. 4B depicts optimization of annealing/extension temperature in the PCR protocol for CAR-T

FIG. 5A depicts ddPCR amplitude plot for CAR-T standard curve at CAR-T concentrations A01, B01, . . . H01 along with a blank sample C02. FIG. 5B depicts ddPCR amplitude plot for alpha actin A01, B01, . . . H01 along with a blank sample C02. FIG. 5C depicts a standard curve for CAR-T cells in the gDNA prepared from mouse blood. FIG. 5D depicts the intra- and inter-assay precision and accuracy of the ddPCR response in the blood QC samples.

FIG. 6A shows raw data obtained after performing a cellular kinetics assay using a ddPCR. ddPCR generates raw data in terms of copy number per microliter of the reaction mixture used. FIG. 6B depicts a formula for conversion of cellular kinetics unit from copy number per microliter of the reaction mixture to copy number per microgram gDNA. FIG. 6C illustrates a formula for conversion of copy number per microliter of reaction to copy number per microliter blood.

FIG. 7 depicts a workflow for evaluation of gDNA extraction recovery (%) from a biological sample. The workflow depicts determination of a gDNA recovery rate (%) by spiking a known amount of a control gene before and after the gDNA extraction process. For CAR-T cells, the workflow depicts the determination of a gDNA recovery rate (%) by spiking a known amount of CAR-T transgene prior to the gDNA extraction process and then by comparing the copy number with a theoretical copy number of CAR-T cells.

FIG. 8A depicts CAR-T cells cellular kinetics measurements by ddPCR in human colorectal tumor bearing NSG mice for 30 days post-administration of CAR-T cells as expressed in terms of copy number per microgram of gDNA. FIG. 8B depicts CAR-T cells cellular kinetics measurements by ddPCR in human colorectal tumor bearing NSG mice for 30 days post-administration of CAR-T cells as expressed in terms of copy number per microliter of mouse blood. FIG. 8C depicts CAR-T cells cellular kinetics measurements by flow cytometry in human colorectal tumor bearing NSG mice for 30 days post-administration of CAR-T cells as expressed in terms of CD3+ CAR-T cells per microliter of mouse blood.

FIG. 9A depicts CAR-T cells cellular kinetics measurements by ddPCR in a human colorectal tumor bearing female NSG mice for 30 days post-administration of CAR-T cells as expressed in terms of CAR-T copy number per microgram of gDNA normalized by actin. FIG. 9B depicts CAR-T cells cellular kinetics measurements by ddPCR in a human colorectal tumor bearing female NSG mice for 30 days post-administration of CAR-T cells as expressed in terms of CAR-T copy number per milligram of tumor tissue.

FIG. 10A depicts comparison of gDNA concentration measurement by a spectrophotometer and a reference gene detection by ddPCR. FIG. 10B depicts expansion of CAR-T cells over 30 days post-administration in terms of CAR-T transgene copy number per microgram gDNA normalized by alpha actin as measured by a ddPCR and a spectrophotometer.

### DEFINITIONS

Administering: As used herein, the terms “administer,” “administering,” or “introducing” are used interchangeably in the context of delivering therapeutic cells, for example, chimeric antigen receptor T (CAR-T) cells, into a subject, by a method or route which results in delivery of such cells. Various methods are known in the art for administering cells, including for example intravenously, subcutaneously or transdermally. Cells can be administered with or without a carrier.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antigen-Specific Targeting Domain: “antigen-specific targeting domain” provides the chimeric antigen receptor (CAR) with the ability to bind to a target antigen of interest. In some embodiments, the antigen-specific targeting domain targets an antigen of clinical interest against which it would be desirable to trigger an effector immune response that results in tumor killing. The antigen-specific targeting domain may be any protein or peptide that possesses the ability to specifically recognize and bind to a biological molecule (e.g., a cell surface receptor or tumor protein, or a component thereof). The antigen-specific targeting domain includes any naturally occurring, synthetic, semi-synthetic, or recombinantly produced binding partner for a biological molecule of interest.

Illustrative antigen-specific targeting domains include, for example, antibodies or antibody fragments or derivatives, extracellular domains of receptors, ligands for cell surface molecules/receptors, or receptor binding domains thereof, and tumor binding proteins.

In some embodiments, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, such as, e.g., a chimeric antigen receptor (CAR) or a T cell receptor (TCR), which is incorporated into the cell's genome.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Adoptive cell therapy (“ACT”) refers to an infusion into patients of autologous or allogeneic cells to treat disease. Various cell types can be used for ACT-based therapies, such as B-cells, T cells, NK-cells, monocytes, progenitor cells, or cell lines. The progenitor cells can be isolated directly from a patient or from a non-patient donor. The progenitor cells include, for example, adult stem cells and pluripotent cells such iPSCs derived from a patient or non-patient donor. In some embodiments, the methods described herein can be used to assess the cellular dynamics of cells that have been introduced into a subject in need thereof. Such determination can then be used, for example, to adjust ACT dosing in a patient.

Chimeric Antigen Receptor (CAR): As used herein, the term “chimeric antigen receptor” or “CAR” engineered receptors which can confer an antigen specificity onto cells (for example NK cells, T cells such as naive T cells, central memory T cells, effector memory T cells or combinations thereof). CARs are also known as artificial T-cell receptors, chimeric T-cell receptors or chimeric immunoreceptors. In some embodiments, the CARs of the invention comprise an antigen-specific targeting domain, an extracellular domain, a transmembrane domain, optionally one or more co-stimulatory domains, and an intracellular signaling domain.

Droplet Digital Polymerase Chain Reaction (ddPCR): As used herein, ddPCR is a method that allows for absolute nucleic acid quantification based on single-molecule PCR. By utilizing microfluidic technology or other droplet generation methods, a large amount of diluted nucleic acid solution is dispersed into micro-wells or droplets. Ideally, the number of target nucleic acid per micro-well or per droplet is less than or equal to one. After amplification, the reactor with the target nucleic acid molecule gives a positive fluorescent signal, and the reactor without the target template shows negative fluorescent signal. Based on the relative ratio and the volume of the reactors, the nucleic acid concentration of the original solution can be derived. Unlike traditional quantitative PCR, ddPCR can achieve absolute quantification of the starting DNA template by direct counting numbers and Poisson correction analysis.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

T cell: As used herein “T cell” is a lymphoid cell defined by its marker expression and function/activity. For example, in humans a T cell expresses CD3.

Single-chain Fv antibody” or “scFv” refers to an engineered antibody that includes a light chain variable region and a heavy chain variable region connected to one another directly or via a peptide linker sequence.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Suffering from: An individual who is “suffering from” a disease, disorder, and/or condition has been diagnosed with or displays one or more symptoms of the disease, disorder, and/or condition. The disease may include cancer, for example, lymphoma and leukemia.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

The present disclosure describes strategies for determining in vivo cellular kinetics and biodistribution of a cell of interest. The characterization of the in vivo cellular kinetics and biodistribution of cells of interest is important with respect to use of cells in adoptive cell therapies. For example, such characterization can be applied to determining toxicity, non-clinical and clinical efficacy of administered chimeric antigen receptor (CAR-T) cells. A well-defined relationship between the number of CAR-T cells administered and CAR-T exposure has not been demonstrated. As such, reliable and reproducible quantitative methods, such as those described herein allow for assessment of various kinetic and biodistribution aspects of administered adaptive cell therapies. Information obtained by using the methods described herein can be used, for example, in the determination of dose selection, administration route and dosing schedule for adoptive cell therapies.

The current gold standard for measuring cellular kinetics is a qPCR-based assay that is used to detect the transgene of CAR-T cells. The FDA draft guidance recommended that the assay should have a demonstrated limit of quantification (LOQ) of ≤50 copies of product per 1 μg genomic DNA (gDNA). The methods described herein provide for a reliable assay that meets and exceeds the FDA LOQ recommendations. The methods described herein can be used to determine both “copy number/μL blood, copy number/mg tissue” and “copy number/μg genomic DNA” at one time of testing.

In some embodiments, the LOQ of the present method is ≤50 copies of product per 1 μg genomic DNA (gDNA). In some embodiments, the LOQ of the present method is ≤40 copies of product per 1 μg genomic DNA (gDNA). In some embodiments, the LOQ of the present method is ≤30 copies of product per 1 μg genomic DNA (gDNA).

The transgene copy number normalized by gDNA input (copy/μg gDNA) is the conventional unit to normalize the gDNA extraction variability during sample preparation process. However, in cases where adoptive cell transfer cells, such as CAR-T cells, expand significantly following administration in vivo and dominate the majority of gDNA extracted from a tissue sample, the apparent transgene copy number with copy/μg gDNA unit will underestimate the actual CAR-T cell expansion. The transgene copy number normalized by blood volume (copy/μL blood) may complement the drawback of copy/μg gDNA unit as the copy/μL blood unit is not affected by the individual difference of gDNA content in blood. The methods provided herein, overcome the limitations of prior methods, as the methods described herein provide a strategy for assessing cellular kinetics and biodistribution for adoptive cell therapy by using a droplet digital PCR (ddPCR) platform with an absolute quantification including automated gDNA extraction process to generate the data with regulatory compliant copy/μg gDNA unit and further unit conversion to copy/μL blood by measuring gDNA extraction recovery. The use of ddPCR has various advantages over other quantification methods, for examples, ddPCR allows for absolute quantification, and as such, no calibration standard is necessary. Furthermore, ddPCR is able to detect rare targets in complex backgrounds, and is more tolerant to PCR inhibitors. Given the advantages of ddPCR, amplification efficiency plays a smaller role in the results than in in other quantification methods such as with qPCR.

In some embodiments, the methods described herein broadly involves two steps: (1) determining copy numbers of the cell of interest per microliter of a reaction mixture/solution using ddPCR, and (2) converting copy numbers of the cell of interest per microliter to copy number per microliter of blood by evaluation of gDNA extraction recovery (%). As described herein, in some embodiments, the methods of the present invention does not involve a pre-isolation step, or sorting step. Accordingly, presorting of a specific cellular population, such as through FACS isolation methods or MACS isolation methods, is not required in the methods described herein.

Various methods are known in the art for measuring gDNA, including for example use of a microvolume spectrophotometer (e.g., Nanodrop), use of fluorescent dye binding assays (e.g., PicoGreen assay), and use of digital droplet PCR (ddPCR). In some embodiments, the methods described herein employ ddPCR as a method of absolute nucleic acid quantification. ddPCR allows for a quantitative assay with higher sensitivity than other methods of quantification, such as qPCR.

The methods described herein can be applied to a cell of interest or to any cellular material that is administered to a human or animal. In some embodiments, the cell of interest has a therapeutic purpose, such as adoptive cell therapy. In some embodiments, cell of interest has diagnostic purpose. Cellular material includes for example, nucleic acids, such as DNA or RNA-based vectors. Accordingly, in some embodiments, the pharmacokinetics of administered cells, e.g., adoptively transferred cells are determined using the methods described herein to assess the availability, e.g., bioavailability of the administered cells.

In some embodiments, cells include allogenic cells, autologous cells, and xenogeneic cells. In some embodiments, cells include stem cells. In some embodiments, the stem cells includes human embryonic stem cells, neutral stem cells, mesenchymal stem cells, and hematopoietic stem cells. In some embodiments, cells are immune cells. In some embodiments, cells are T-cells. In some embodiments, T-cells are chimeric antigen receptor T cells. In some embodiments, cells are natural killer cells.

In some embodiments, a method is provided for determining copy numbers of the cell of interest in a biological sample. In some embodiments, the copy number of the cell of interest is determined per microliter of a reaction mixture, the method includes extracting gDNA from a biological sample and running ddPCR on the extracted gDNA using the cell of interest and a reference gene. The biological sample can be any biofluid or tissue sample. For example, biofluids include urine, stool/feces, ascites fluid, saliva, sputum, synovial fluid, and cerebrospinal fluid (CSF). Tissue samples include, for example, kidney or liver among others.

In some embodiments, a biological sample includes any biofluid or tissue sample. In some embodiments, a biological sample is human or animal biological sample. In some embodiments, the biological sample is a tumor mass. In some embodiments, the tumor is a solid tumor.

In some embodiments, the gDNA is extracted using an automated process. In some embodiments, the gDNA is extracted manually. In one embodiment, gDNA is extracted with MagMAX™ DNA Multi-Sample Ultra 2.0 Kit.

Use of automated genomic DNA extraction process enables highly efficient and reproducible genomic DNA extraction. In this manner, there is no need to count cell number for each sample, which in turn results in a quicker, less labor intensive method which is applicable to both nonclinical and clinical settings.

There are several bioanalytical methods available for detecting and measuring copy number of cells in biomatrices, such as for example, microvolume spectrophotometer (e.g., Nanodrop), fluorescent dye binding assays (e.g., PicoGreen assay), and digital droplet PCR (ddPCR). Accordingly, in some embodiments, cell numbers are measured using quantitative polymerase chain reaction (qPCR). In some embodiments, cell numbers are measured using flow cytometry. In some embodiments, cell numbers are measured using a fluorescence and/or radiolabeled imaging. In some embodiments, cell numbers are measured using a ddPCR.

As describes earlier, ddPCR provides copy number of a transgene in terms of copy number per microliter of the reaction mixture. In some embodiments, the unit of copy number per microliter of the reaction mixture is converted into copy number per microgram of the gDNA. In some other embodiments, the unit of copy number per microliter of the reaction mixture is converted into copy number per microliter of blood. In some other embodiments, the unit of copy number per microliter of the reaction mixture is converted into copy number per microgram of tissue.

### Converting Copy Number per Microliter of Reaction Mixture to Copy Number per Microgram of gDNA

In some embodiments, cellular kinetics unit copy number per microliter of the reaction mixture is converted into copy number per microgram of the gDNA, a FDA regulatory compliant unit, by using the below Formula I:

$$\frac{copy}{μg gDNA} = \frac{\frac{transgene copy}{μL reaction} \times 1,000,000}{\frac{reference gene copy}{μL reaction} \times \frac{3.3 pg}{copy}}$$

wherein copy represents copy number; and reaction represents reaction mixture or solution, pg represents picogram, and

$$\frac{3.3 pg}{copy} = \frac{3 \times 1 {0}^{9} bp}{copy} \times \frac{660 g}{mol bp} \times \frac{1 mol bp}{6.023 \times 1 {0}^{23} bp} \times \frac{1 \times 1 {0}^{12} pg}{g}$$

wherein bp represents base pair, mol represents mole, g represents gram, 6.023×10²³ represents an Avogadro number, 3.0×10⁹ base pair represents the size of a human genome, and 660 g/mol bp represents the molecular weight of each base pair.

In some embodiments, the unit copy number per microgram of gDNA (i.e., copy/μg gDNA) is generated by the ratio of a gene of interest, for example, CAR-T transgene copy number normalized by a single copy reference gene, for example, such as alpha actin copy number. Although there are two copies of the target sequence in majority of human cells, the target sequence is present in a single copy in the human haploid genome. The haploid human genome size is 3.0×109 base pair. By taking the molecular weight of a base pair (i.e., 660 g/mol) and Avogadro's number (1 mol/6.023×1023) into account, the base pair number per genome copy number can be converted to an amount of DNA per genome copy number, as shown in above formula.

Actin or α-actin (the reference gene) is known to have one copy per haploid genome, and one haploid genome is about 3.3 pico gram, meaning alpha actin is characterized to have one copy per 3.3 pg of gDNA. Other reference genes can be used, and include for example, RNase P, gamma tubulin 1, or GAPDH.

### Converting Copy Number per Microliter of Reaction Mixture to Copy Number per Microliter of Blood or Copy Number per Milligram of Tissue

In some embodiments, cellular kinetics unit copy number per microliter of the reaction mixture is converted into copy number per microliter of blood by using the below

$$\frac{copy}{μ L blood} = \frac{transgene copy}{μ L of PCR reaction buffer} \times \frac{total μ L of PCR reaction buffer}{μ L of eluate used} \times \frac{total μ L of eluate}{total μ L of blood used} \times \frac{1}{genomic DNA recovery ( \% )} \qquad Formula II$$

In some embodiments, cellular kinetics unit copy number per microliter of the reaction mixture is converted into copy number per milligram of tissue by using the below

$$\frac{copy}{mg tissue} = \frac{transgene copy}{μ L of PCR reaction buffer} \times \frac{total μ L of PCR reaction buffer}{μ L of eluate used} \times \frac{total μ L of eluate}{total mg of tissue used} \times \frac{1}{genomic DNA recovery ( \% )} \qquad Formula III$$

In some embodiments, to covert copy number per microliter of the reaction mixture into either copy number per microliter of blood or copy number per milligram of tissue requires: a) taking into accounts of all biometrices used, and b) the genomic DNA recovery (%) or gDNA extraction recovery (%) from a biological sample.

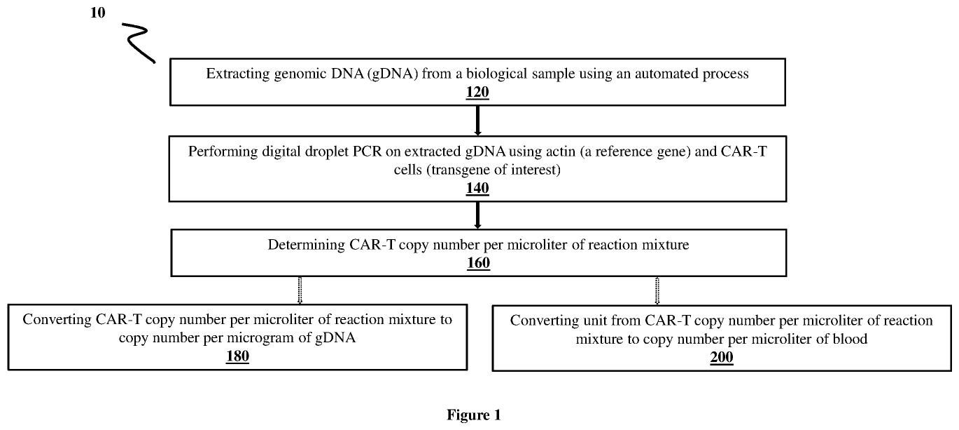

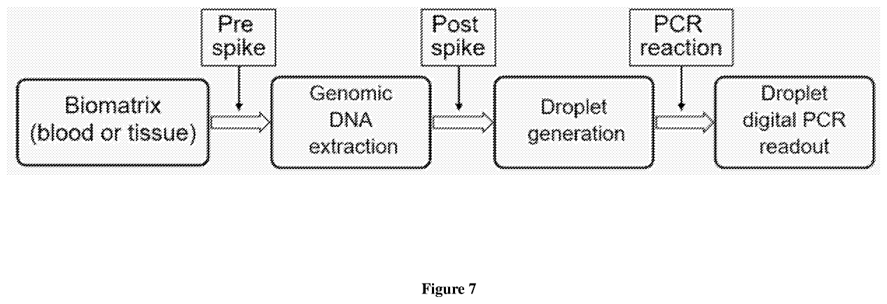

gDNA extraction recovery (%) represents the efficiency of the process, preferably an automated process, of extraction of gDNA from the biological sample, as shown in step 120 of FIG. 1. In some embodiments, gDNA recovery (%) is determined by comparing a known amount of gene, for example spkHIS or spkLEU, at before and after genomic DNA extraction process, as shown in FIG. 7.

In some embodiments, a known amount of a reference gene is used to spike a biomatrix, for example a blood sample or a tissue sample/homogenate, immediately before (pre-spike) and after (post-spike) the gDNA extraction process as shown in the workflow at FIG. 7. gDNA recovery (%) is calculated by comparing the ddPCR outputs (in terms of copy number/μL of reaction mixture) for pre-spike and post-spike events. In some embodiments, pre-spike and post-spike steps are performed in two separate ddPCR runs by maintaining all other parameters identical.

In some embodiments, gDNA recovery (%) for a reference gene (exo-gene) is about 60% to about 100% as shown in Table 1 for exo-genes HIS3 and LEU2. In some embodiments, gDNA recovery (%) for a reference gene ranges from 100%-65%, 100%-70%, 100%-75%, 100%-80%, 100%-85%, 100%-90%, and 100%-95%. In some embodiments, gDNA recovery (%) for a reference gene is less than 100%, less than 95%, less than 90%, less than 85%, less than 80%, less than 75%, less than 70%, less than 65%, and less than 60%. In some embodiments, gDNA recovery (%) for a reference gene is greater than 60%, greater than 65%, greater than 70%, greater than 75%, greater than 80%, greater than 85%, greater than 90%, greater than 95%.

In some embodiments, importantly, gDNA recovery (%) in tissue samples is approximately 95-100% as shown in Table 2. In some embodiments, importantly, gDNA recovery (%) rate in tissue samples is ranges from 100%-80%, 100%-85%, 100%-90%, and 100%-95%. In some embodiments, gDNA recovery (%) for a reference gene is less than 100%, less than 95%, less than 90%, less than 85%, and less than 80%. In some embodiments, gDNA recovery (%) for a reference gene is greater than 80%, greater than 85%, greater than 90%, greater than 95%, greater than 100%.

In some other embodiments, gDNA recovery (%) is determined by spiking a known amount of transgene of interest prior to the gDNA extraction step and by comparing the resultant copy number obtained from ddPCR readout with the theoretical copy number following the similar steps shown in FIG. 7.

$$Theoretical copy number = \frac{Cell number}{μL} \times \frac{2 copies}{cell} \times \frac{1}{transduction efficiency ( \% )} \times \frac{μL of PCR reaction buffer}{total μL of PCR reaction buffer}$$

In some embodiments, gDNA recovery (%) for a transgene of interest is about 50% to about 100% as shown in Table 2 for example a CAR-T cell. In some embodiments, gDNA recovery (%) for a reference gene ranges from 100%-60%, 100%-65%, 100%-70%, 100%-75%, 100%-80%, 100%-85%, 100%-90%, and 100%-95%. In some embodiments, gDNA recovery (%) for a reference gene is less than 100%, less than 95%, less than 90%, less than 85%, less than 80%, less than 75%, less than 70%, less than 60%, and less than 50%. In some embodiments, gDNA recovery (%) for a reference gene is greater than 50%, greater than 60%, greater than 65%, greater than 70%, greater than 75%, greater than 80%, greater than 85%, greater than 90%, greater than 95%, greater than 100%.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1. Strategy for Performing Cellular Kinetics Assay of CAR-T Cells Using ddPCR

This example illustrates a strategy for performing a cellular kinetics assay of CAR-T cells using ddPCR. The strategy broadly involves two steps: (1) use of a ddPCR to detect both CAR-T cells (i.e., a transgene of interest) and a single copy reference gene (in this example alpha actin) so as to normalize the genomic DNA input, and (2) conversion of unit from copy number per microgram of gDNA to copy number per microliter of blood by evaluation of gDNA extraction recovery (%).

Step 1 enables more accurate quantification of CAR-T cells in terms of copy number per microgram gDNA, which is a FDA regulatory compliant unit. Furthermore, step 1 has negligible effect of matrix characteristics that makes ddPCR quite useful for measuring cellular kinetics and biodistribution without the need of a standard curve.

However, the unit of copy number per microgram of gDNA has limitations in conducting a pharmacokinetics (PK) analysis. Step 2 evaluates gDNA extraction recovery (%) which is important for converting copy number per microliter of reaction mixture to copy number per microliter of blood, and enables a PK or similar analysis.

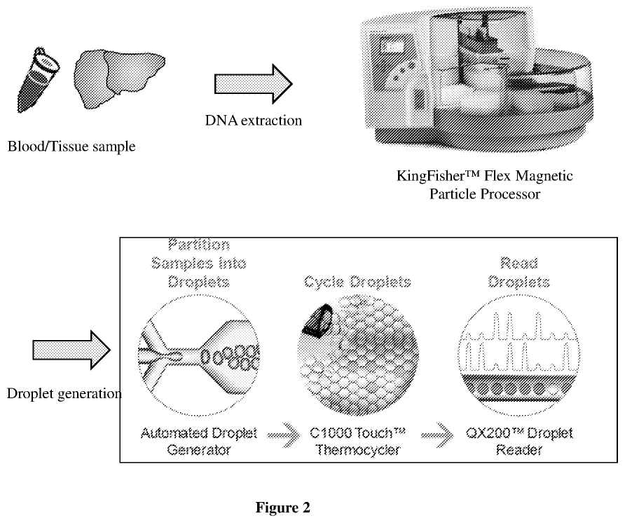

FIG. 1 depicts a strategy 10 for conducting a cellular kinetics assay of CAR-T cells using a ddPCR in more detail. The strategy 10 includes extracting 120 genomic DNA (gDNA) from a biological sample (a blood/tissue sample) using an automated process (as described below in FIG. 2), performing 140 ddPCR on extracted gDNA using alpha actin (a single copy reference gene) and CAR-T (the transgene of interest), determining 160 CART copy number per microliter of reaction mixture, optionally converting 180 CART copy number per microliter of reaction mixture to copy number per microgram of gDNA, or directly converting 200 copy number per microliter of reaction mixture to a copy number per microliter of blood by evaluating gDNA extraction recovery (%) and normalizing for different volumes.

### Workflow for Cellular Kinetics and Tissue Distribution Assays Using ddPCR

FIG. 2 depicts a workflow for cellular kinetics and tissue distribution assays using ddPCR. The workflow involves several steps including, but not limited to, gDNA extraction from a biological sample, droplet generation, PCR reaction, and cell number determination as explained below in more detail.

### Example 2. gDNA Extraction From Mouse Blood or Tissue Sample

This example illustrates gDNA extraction from mouse blood or tissue samples/homogenates. gDNA was extracted with MagMAX™ DNA Multi-Sample Ultra 2.0 Kit with some minor modifications. gDNA extraction was performed in presence of Proteinase K and Ambion™ RNase A (5 μL each, affinity purified, Thermo Fisher Scientific) to ensure negligible RNA contamination. Tissue samples were homogenized in a 9-fold volume of PBS containing 15 mM EDTA with gentleMACS Octo Dissociator in the mode of RNA-01-01. A binding enhancer solution, homogenate solution and proteinase K were mixed well in a plate, and then incubated the plate at 65° C. for overnight. The gDNA extraction process was automated with KingFisher™ Flex system using the sample processing programs (MagMAX_Ultra2_200 μL_Flex and MagMAX_Ultra 2_Tissue_V) as per manufacture's protocol.

The gDNA quality from each extracted sample was monitored by NanoDrop 2000 spectrophotometer with the ratio of absorbance at 260 nm and 280 nm. The extracted gDNA from mouse blood and tumor tissue showed ratios of the absorbance at 260 and 280 nm (A_{260/280}) of 1.92±0.10 and 1.93±0.04 (n=45 each), respectively which indicated high purity of gDNA with minimal protein and/or RNA contamination as also confirmed by an agarose gel electrophoresis.

### Example 3. Determination of Transgene Copy Number in CAR-T Cells by ddPCR

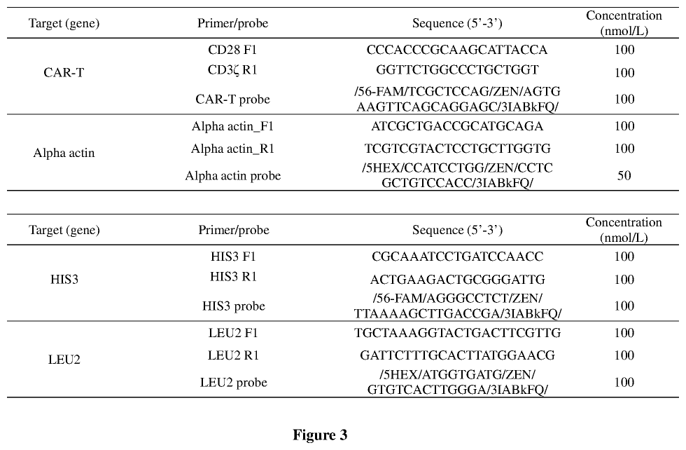

Droplet generation and cell number determination were performed using ddPCR. A TaqMan PCR reaction mixture (20 μl) comprising 2× ddPCR supermix for probes (no dUTP), 20× primer and probes (as shown in FIG. 3), 40× restriction enzyme (XbaI, EcoRI-HF, 10 U/μL, New England Biolabs, Ipswich, MA) and the template (4 μL) was prepared, and subjected to droplet generation. FIG. 3 depicts selection of primers for CAR-T cells. The CD28 co-stimulatory and CD3ζ signal transduction domain specific primers and probe were synthesized and used to amplify the junction between CD28 and CD3ζ. Upon droplet generation, emulsified samples were transferred to a 96-well PCR plate by QX200™ Droplet Generator. The head-sealed plate was then placed on the C1000 Touch™ Thermal Cycler, and the target specific gene was amplified as follows. The thermal cycling condition was 95° C. for 5 min (1 cycle), 40 cycles at 95° C. for 0.5 min and at 63° C. for 2 min and hold at 4° C. After thermal cycling, the positive and negative fluorescent droplets were counted in the QX100 Droplet Reader, and genomic copy numbers were calculated with the Quanta Soft program (Bio-Rad, Hercules, CA). For tissue homogenates, quantitative ddPCR was performed on 200 ng tissue derived gDNA per reaction.

Droplet generation included partitioning (that kept concentration of molecules per unit volume constant) of extracted gDNA into tiny droplets. In some embodiments, droplet generation process produces about 20,000 droplets of approximately one nanoliter size. The plurality of droplets are then cycled in a thermocycler, and finally droplets are read in a droplet reader to provide a read out.

The distribution of transgene target sequence in the droplet partitions is described by Poisson distribution. The absolute quantification of the transgene target was based on the ratio of positive signal against all droplet partitions at the end of PCR reaction. The concentration of transgene copy number (copy per droplet) is determined as follows.

$$Concentration = - \frac{ln ( \frac{N umber of negative droplets}{Number of total droplets} )}{{V}_{droplet}}$$

ddPCR enables absolute quantification of gDNA without any need of a calibration curve, and ddPCR consistently results in lower variation in cell number. ddPCR is more tolerant to PCR inhibiters, and also enables detection of rare targets in complex backgrounds.

In some embodiments, gDNA extraction and ddPCR steps are conducted separately. In one embodiment, all steps involved in the workflow as shown in FIG. 2 are conducted using a KingFisher™ Flex Magnetic Particle Processor followed by ddPCR readout. In some embodiment, the workflow is performed in an automated process.

### Example 4. Evaluation of Performance of ddPCR in Cellular Kinetics Assay

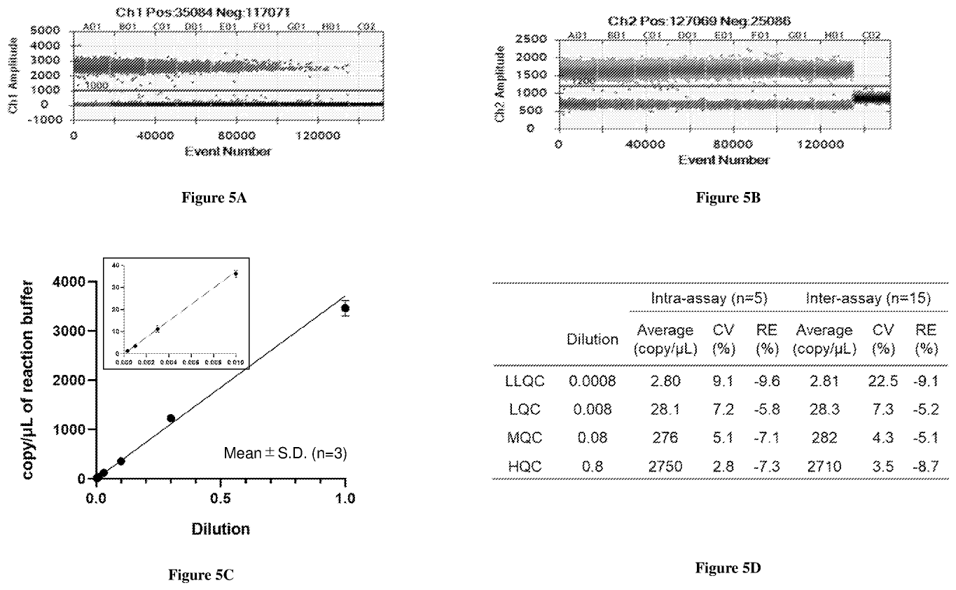

The ddPCR assay performance was evaluated based on the selectivity, linearity, intra- and inter-assay precision, accuracy and robustness. The specificity of the ddPCR assay was evaluated by the no template control (NTC, water) which monitors contamination and/or primer-dimer formation which could cause false positive results. To evaluate the linearity of the assay, a linear model fitted by least-squares linear regression with weighting factor 1/x² was used to describe the calibration curve. The calibration curve and quality control (QC) samples were prepared in 50 μg/mL of gDNA solution extracted from mouse blood. The x-axis represents the dilution factor (0.0003, 0.001, 0.003, 0.01, 0.03, 0.1, 0.3, 1), and y-axis represents the absolute number of copy/μL reaction buffer, as shown below in FIG. 5C. The QC samples were at the dilution level of 0.0008 (lower limit of quantitation, LLOQ), 0.008 (low-QC, LQC), 0.08 (medium-QC, MQC), 0.8 (high-QC, HQC), respectively, as shown below in FIG. 5D. The assay robustness was evaluated by the repeatability including intra- and inter-assay precision and accuracy. The criteria of the relative error (% RE) of the back-calculated concentrations to the nominal concentrations was set as within ±20% of nominal values (for LLOQ; ±25%). The stability of sample was tested in incurred mouse blood after a single intravenous administration of CAR-T positive cells at a dose of 1.0×10⁶ cells/mouse stored in a freezer set at −80° C. for 2 months.

### Example 5. Duplexing ddPCR Assay Development for Absolute Quantification of CAR-T and Reference Gene

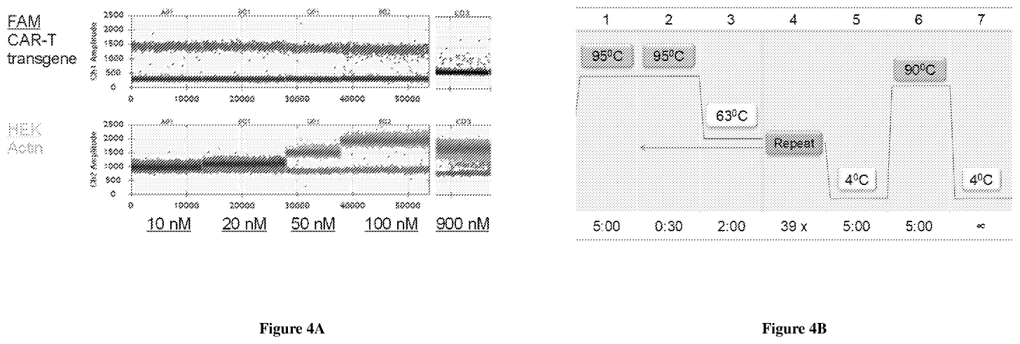

In order to develop a duplexing ddPCR assay for absolute quantification of CAR-T cells and reference gene (preferably a single copy gene on the chromosome), assay conditions including primer concentrations and annealing temperature were first optimized. The optimal condition for alpha actin (a single copy reference gene) primer concentrations (10, 20, 50, 100 and 900 nmol/L) was investigated for duplexing assay of CAR-T transgene and reference gene detection by ddPCR. At 100 nmol/L of each primer, the positive and negative signals were well separated. Lower concentration of primer for alpha actin decreased the quality of separation and higher concentration of each primer tends to interfere the PCR efficiency, as shown in FIG. 4A. The annealing temperature was optimized by carrying out temperature gradient PCR for both CAR-T transgene and reference gene as shown in FIG. 4B.

A duplexing ddPCR assay for CAR-T transgene and a single copy reference gene was designed by a combination of primers with amplicon where the junction of CD28/CD3ζ and amplicon of the single copy reference gene were detected by 6-FAM and HEX-labeled probes, respectively. The primer concentrations for CAR-T transgene and the single copy reference gene were 100 nmol/L, and the target specific annealing/extension temperature in the PCR reaction was set at 63° C. for 2 min. The maximum amount of gDNA input in the ddPCR reaction was set at 200 ng gDNA (4 μL of 50 μg/mL gDNA solution in PCR reaction) in order to get the single copy reference gene copies within the dynamic range of detection. As the reference gene exists as a single copy gene per haploid human genome, 3.3 pg gDNA contained single copy of reference gene. The 200 ng gDNA input in the PCR reaction generated 3293±362 copy/μL reaction of reference genes (theoretical single copy reference gene copy number: 3030 copy/μL reaction) which is well below the upper limit of quantification range (i.e., 5000 copy/μL), as shown in FIG. 5B, in the ddPCR system.

### Example 6. Qualification of Analytical Techniques Used in CAR-T Cellular Kinetics Assay

This example illustrates the qualification of techniques/methods used in cellular kinetics assay of CAR-T cells.

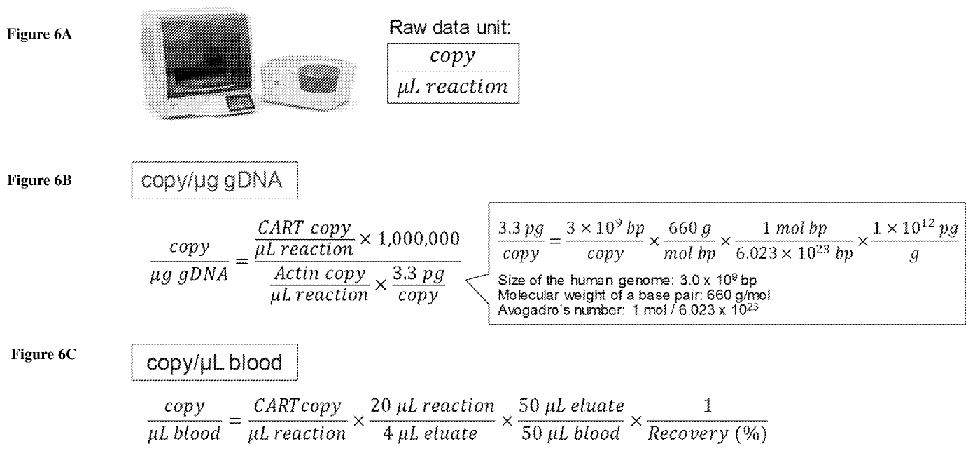

FIG. 5A depicts the ddPCR amplitude plot for the CAR-T transgene standard curve (A01, B01, . . . H01) and blank samples (C02), and FIG. 5B depicts the ddPCR amplitude plot for the reference gene and blank samples (C02). The ddPCR amplitude plots for the CAR-T transgene and single copy reference gene show clear separations between the positive (blue for CAR-T, green for single copy reference gene) and negative droplets (black) with only few interface droplets confirming a specific and efficient PCR reaction. The selectivity of the ddPCR assay for the CAR-T transgene and reference gene was also assessed by performing measurement of a no template control sample, shown as C02 in FIG. 5A and FIG. 5B), confirming the selectivity of the ddPCR assay. FIG. 6A and FIG. 6B further confirms that there was negligible contamination and/or primer-dimer formation during the ddPCR assay.

FIG. 5C depicts a standard curve plotted by diluting the CAR-T plasmid DNA in 50 μg/mL of gDNA extracted from mouse blood. The standard curve is linear in the calibration range from the dilution level of 0.0003 to 1 (1.27-3460 copy/μL reaction) and is fitted with a weight of 1/x² (y=3709×0.129, R²=0.9655). FIG. 5D depicts inter- and intra-assay precision and accuracy of the response in blood QC samples. The precision (% CV) and accuracy (% RE) in intra- and inter-assays were within 22.5% and within −9.6-−5.1% of nominal values, respectively at the level of the dilution of 0.008, 0.008, 0.08, 0.8 prepared from the upper limit of quantification (ULOQ) sample. The limit of detection copy number was 0.3 copy/μL reaction. The stability of sample was tested in incurred mouse blood after a single intravenous administration of CAR-T positive cells at a dose of 1.0×10⁶ cells/mouse stored in a freezer set at −80° C. for 2 months.

### Example 7. Generation of Cellular Kinetics Data in Terms of Copy Number per Microgram gDNA and Copy Number per Microliter of Blood

This example illustrates generation of cellular kinetics of CAR-T cells in terms of copy number per microgram gDNA and copy number per microliter of blood using ddPCR.

FIG. 6A shows raw data unit obtained after performing a cellular kinetics assay using a ddPCR in one embodiment. ddPCR generates raw data in terms of copy number per microliter of the reaction mixture used. However, the FDA regulatory compliant unit to measure cellular kinetics is copy number per microgram gDNA, and therefore, conversion of cellular kinetics unit from copy number per microliter of the reaction mixture to copy number per microgram gDNA is required.

FIG. 6B illustrates a formula for conversion of cellular kinetics unit from copy number per microliter of the reaction mixture to copy number per microgram gDNA. The cellular kinetics data with the unit of copy/μg gDNA was generated by the ratio of CAR-T and a single copy reference gene (here alpha actin) copy number normalized by the number of DNA per genome copy number. Since there are two copies of the target sequence in majority of human cells, the target sequence is present in a single copy in the human haploid genome. The haploid human genome size is 3.0×109 base pair. By taking the molecular weight of a base pair (660 g/mol) and Avogadro's number (1 mol/6.023×1023) into account, the base pair number per genome copy number was converted to an amount of DNA per genome copy number.

As described earlier, copy number per microgram gDNA unit also has limitations in terms of PK analysis, and therefore, copy number per microliter of reaction mixture needs to be converted to copy number per microliter blood. FIG. 6C illustrates a formula for conversion of copy number per microliter of reaction to copy number per microliter blood. The unit of copy number per microliter of reaction was converted to copy number per microliter blood by normalizing copy number per microliter of reaction mixture with the volume of PCR reaction buffer, gDNA extraction volume, applied blood volume and gDNA extraction recovery (%).

### Example 8. Determination a Genomic DNA Recovery Rate by Spiking a Known Amount of a Control Gene Before and After the gDNA Extraction Process

This example illustrates a method of evaluation of gDNA extraction recovery (%) or gDNA extraction recovery rate. The method includes spiking a known amount of a control gene or exo-gene (for example, linearized yeast HIS3 or LEU2 plasmid DNA) before and after the gDNA extraction process. The gDNA extract that was spiked with control gene was then subjected to droplet generation, PCR reaction and ddPCR readout steps as shown in FIG. 7. The gDNA recovery was measured by comparison of copy number from pre- and post-spike sample.

In one embodiment, a known amount of exo-gene HIS3 was used to spike gDNA extract before and after the gDNA extraction process. In another embodiment, a known amount of exo-gene LEU2 was used to spike gDNA extract before and after the gDNA extraction process. The ddPCR readout produces copy number per microliter of the reaction mixture for both pre-spike control gene and post-spike control gene. The copy number per microliter for both pre-spike control gene and post-spike control gene were then used to calculate gDNA extraction recovery (%). In order to achieve a consistent gDNA extraction recovery (%), the aforesaid method is repeated with different matrix conditions such as with 1% blood sample, 5% blood sample, 10% blood sample, with 50% blood, and with whole blood. In some embodiments, a matrix or a biological sample is spiked with a control gene at a low quality control (LQC) amount, a medium quality control (MQC) amount, and/or a high quality control (HQC) amount.

Table 1 lists copy number per microliter of reaction mixture for both pre-spike and post-spike exo-gene obtained under different matrix and quality control conditions. The left panel, labelled as (A) represents exo-gene HIS3, whereas the right panel, labelled as (B) represents exo-gene LEU2. gDNA extraction recovery (%) for both HIS3 and LEU2 under different conditions were calculated, as shown in Table 1. The exo-gene recovery (%) ranged from about 60% to about 100% suggesting the difficulty in consistently obtaining the 100% recovery, even with the fully automated gDNA extraction process. It is believed that lower content of white blood cells (i.e., lymph depleted condition) does not significantly affect the gDNA extraction recovery (%). In contrast, the exo-gene recovery (%) was 95-100% in 10% liver, tumor and kidney homogenate, as shown in Table 2.

TABLE 1
gDNA extraction recovery (%) for exo-genes HIS3 and LEU2 in mouse blood

| | | Exo-gene HIS3 (copy/μL) | | | | Recovery | | Exo-gene LEU2 (copy/μL) | | | | Recovery |
|---|---|---|---|---|---|---|---|---|---|---|---|---|
| | | Pre-spike | | Post-spike | | (%) | | Pre-spike | | Post-spike | | (%) |
| | (A) | Mean | S.D. | Mean | S.D. | Mean | (B) | Mean | S.D. | Mean | S.D. | Mean |
| 1% | LQC | 5.4 | 2.0 | 6.9 | 0.5 | 78.4 | LQC | 9.9 | 3.0 | 9.0 | 1.1 | 109.6 |
| Mouse | MQC | 49.8 | 5.3 | 60.6 | 0.8 | 82.2 | MQC | 80.2 | 22.5 | 80.3 | 2.6 | 99.9 |
| blood | HQC | 430.3 | 21.8 | 540.3 | 12.7 | 79.6 | HQC | 635.0 | 104.9 | 752.7 | 27.7 | 84.4 |
| 5% | LQC | 4.2 | 0.3 | 5.9 | 0.8 | 71.3 | LQC | 7.1 | 1.2 | 8.2 | 1.4 | 86.9 |
| Mouse | MQC | 43.1 | 4.4 | 61.7 | 0.5 | 69.8 | MQC | 64.0 | 5.0 | 83.5 | 3.8 | 76.7 |
| blood | HQC | 385.7 | 8.3 | 568.0 | 8.7 | 67.9 | HQC | 515.0 | 14.4 | 769.3 | 4.5 | 66.9 |
| 10% | LQC | 4.4 | 0.3 | 6.2 | 0.9 | 70.1 | LQC | 5.1 | 1.2 | 7.9 | 0.8 | 63.9 |
| Mouse | MQC | 39.6 | 2.5 | 61.7 | 1.3 | 64.2 | MQdC | 51.0 | 1.1 | 85.6 | 1.4 | 59.5 |
| blood | HQC | 364.7 | 27.7 | 561.0 | 12.0 | 65.0 | HQC | 496.7 | 20.8 | 759.3 | 26.4 | 65.4 |
| 50% | LQC | 5.5 | 0.6 | 5.7 | 0.9 | 96.5 | LQC | 9.0 | 0.9 | 8.0 | 0.7 | 111.6 |
| Mouse | MQC | 59.4 | 2.8 | 62.6 | 1.8 | 94.9 | MQC | 78.0 | 5.1 | 81.7 | 2.7 | 95.4 |
| blood | HQC | 575.3 | 6.8 | 548.0 | 9.6 | 105.0 | HQC | 781.0 | 24.0 | 742.7 | 24.8 | 105.2 |
| 100% | LQC | 8.0 | 0.9 | 6.9 | 0.5 | 115.5 | LQC | 8.9 | 1.0 | 8.3 | 0.6 | 107.7 |
| Mouse | MQC | 67.0 | 2.9 | 60.5 | 0.5 | 110.9 | MQC | 88.7 | 3.1 | 78.2 | 1.5 | 113.3 |
| blood | HQC | 601.3 | 26.6 | 579.0 | 13.5 | 103.9 | HQC | 837.3 | 18.3 | 798.0 | 28.8 | 104.9 |

TABLE 2
gDNA extraction recovery (%) for exo-genes HIS3 and LEU2 in 10% tissue homogenates

| | | Exo-gene HIS3 (copy/μL) | | | | Recovery | | Exo-gene LEU2 (copy/μL) | | | | Recovery |
|---|---|---|---|---|---|---|---|---|---|---|---|---|
| | | Pre-spike | | Post-spike | | (%) | | Pre-spike | | Post-spike | | (%) |
| | (A) | Mean | S.D. | Mean | S.D. | Mean | (B) | Mean | S.D. | Mean | S.D. | Mean |
| 10% | LQC | 6.9 | 0.9 | 7.0 | 0.2 | 98.6 | LQC | 9.3 | 0.7 | 8.6 | 0.7 | 107.2 |
| Liver | MQC | 68.1 | 5.6 | 65.9 | 1.5 | 103.3 | MQC | 89.3 | 3.6 | 87.3 | 4.0 | 102.3 |
| | HQC | 668.0 | 21.2 | 677.3 | 23.6 | 98.6 | HQC | 889.0 | 24.6 | 894.0 | 23.5 | 99.4 |
| 10% | LQC | 7.2 | 0.4 | 6.6 | 1.0 | 108.3 | LQC | 9.1 | 0.8 | 9.1 | 0.6 | 100.3 |
| Tumor | MQC | 65.3 | 5.1 | 66.3 | 2.9 | 98.6 | MQC | 85.4 | 7.3 | 84.6 | 2.1 | 100.9 |
| | HQC | 641.3 | 50.5 | 669.5 | 15.6 | 95.8 | HQC | 861.5 | 79.8 | 880.3 | 12.8 | 97.9 |
| 10% | LQC | 7.6 | 0.6 | 7.4 | 1.3 | 103.1 | LQC | 9.4 | 0.9 | 8.6 | 0.7 | 109.9 |
| Kidney | MQC | 71.8 | 5.0 | 72.3 | 1.5 | 99.3 | MQC | 93.5 | 5.8 | 90.3 | 2.1 | 103.6 |
| | HQC | 686.0 | 19.4 | 684.3 | 6.2 | 100.3 | HQC | 940.0 | 49.6 | 902.5 | 21.5 | 104.2 |

### Example 9. Determination a Genomic DNA Recovery Rate by Spiking a Known Amount of a CAR-T Cells Transgene Prior to the gDNA Extraction Process, and Comparing Resultant Copy Number With a Theoretical Copy Number

This example illustrates a method of determination of gDNA extraction recovery rate by spiking a known amount of a CAR-T cells transgene prior to the gDNA extraction process, and then comparing resultant copy number (i.e., ddPCR readout) with a theoretical copy number (CAR-T cells). The method includes spiking a known amount of a CAR-T cells transgene prior to the gDNA extraction process. The gDNA extract was then subjected to droplet generation, PCR reaction and ddPCR readout steps as shown in FIG. 7. The ddPCR readout produces copy number per microliter of the reaction mixture for CAR-T cells transgene. The copy number per microliter for CAR-T cell transgene is then compared with a theoretical copy number per microliter to calculate gDNA extraction recovery (%). In a low quality control (LQC) condition, 2.0×10⁴ cells/mL were used. In a medium quality control (MQC) condition, 2.0×10⁵ cells/mL were used. In a low quality control (LQC) condition, 2.0×10⁶ cells/mL were used. It was assumed that each cell includes 2 copies and the transduction efficiency was 60%.

Table 3 lists copy number per microliter for pre-spike CAR-T cell transgene obtained under different matrix and quality control conditions. gDNA extraction recovery (%) for CAR-T cell transgene under different conditions was calculated and is shown in Table 3. The CAR-T cell recovery (%) ranged from about 50% to about 100%, similar to exo-genes.

TABLE 3
gDNA extraction recovery (%) for CAR-T cells in mouse blood

| | | CART cells (copy/μL) | | | Recovery |
|---|---|---|---|---|---|
| | | Pre-spike | | | (%) |
| | (C) | Mean | S.D. | Theoretical | Mean |
| 1% | LQC | 12.7 | 1.1 | 13.2 | 96.0 |
| Mouse | MQC | 93.5 | 14.8 | 132.2 | 70.7 |
| blood | HQC | 1307.0 | 29.0 | 1322.3 | 98.8 |
| 5% | LQC | 10.3 | 1.6 | 13.2 | 78.1 |
| Mouse | MQC | 92.8 | 12.1 | 132.2 | 70.2 |
| blood | HQC | 772.7 | 58.2 | 1322.3 | 58.4 |
| 10% | LQC | 7.7 | 0.6 | 13.2 | 58.2 |
| Mouse | MQC | 55.8 | 3.2 | 132.2 | 42.2 |
| blood | HQC | 700.3 | 75.4 | 1322.3 | 53.0 |
| 50% | LQC | 13.8 | 3.0 | 13.2 | 104.4 |
| Mouse | MQC | 135.3 | 18.2 | 132.2 | 102.3 |
| blood | HQC | 1170.7 | 276.3 | 1322.3 | 88.5 |
| 100% | LQC | 10.2 | 1.0 | 13.2 | 77.4 |
| Mouse | MQC | 145.3 | 4.0 | 132.2 | 109.9 |
| blood | HQC | 1390.3 | 146.3 | 1322.3 | 105.1 |

### Cellular Kinetics Measurements in Tumor Bearing Mice

### Example 10. Cellular Kinetics in Female NSG Mice Bearing Xenograft as Measured by ddPCR and Flow Cytometry

This example illustrates cellular kinetics of CAR-T cells after a single intravenous injection of CAR-T positive cells to female NSG mice bearing xenograft. A suspension of human colorectal cancer model cell line was subcutaneously injected into the flank of female NOD SCID Gamma (NSG) mice at 2.0×10⁶ cells/mouse. Animals were sorted into treatment groups (n=3) having an initial tumor volume of approximately 150 mm³. The animals were given a single intravenous administration of CAR-T positive cells at a dose of 1.0×10⁶ CAR positive T cells/mouse. Mice were sacrificed at designated time points—1 h, day 1, 3, 7, 10, 14, 21 and 28 post-administration, and blood and tumor samples were harvested. Tumor volume and body weight were measured before the sample collection. Samples were frozen and stored under −80° C. until sample analysis. CAR-T cells kinetics was measured in blood for thirty days post-injection. The cellular kinetics was determined both in terms of copy number per microgram gDNA and copy number per microliter of blood.

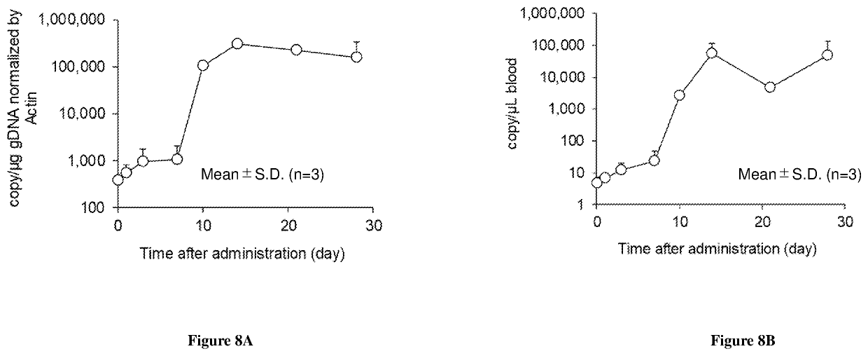

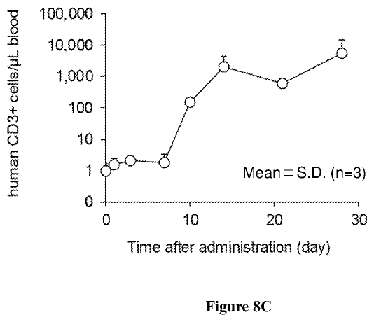

FIG. 8A depicts cellular kinetics of CAR-T cells expressed in terms of copy number per microgram of gDNA as measured by ddPCR. gDNA of CAR-T transgene was normalized using alpha actin, a single copy reference gene. FIG. 8B depicts cellular kinetics of CAR-T cells expressed in terms of copy number per microliter of blood as measured by ddPCR. FIG. 8C depicts cellular kinetics of CAR-T cells expressed in terms of hCD3⁺ cells per microliter of blood as measured by a flow cytometry assay.

As shown in FIG. 8A and FIG. 8B, immediately after the administration of CAR-T cells, the CAR-T transgene level became low due to a quick distribution of cells throughout the peripheral blood, bone marrow, lung, and other tissues. However, about seven days post-administration of cells, a rapid in vivo expansion of CAR-T cells in blood was observed. A similar cellular kinetic profile was also observed when the CAR-T cells expansion were measured using a flow cytometry assay as shown in FIG. 8C. The cellular kinetics profile of CAR-T cell expansion was well correlated between the transgene level by ddPCR and the CAR-T expression level by flow cytometry. Notably, the transgene level expressed in copy number per microgram of gDNA, as shown in FIG. 8A, underestimated the CAR-T cell expansion as compared to the transgene level expression in terms of copy number per microliter of blood, as shown in FIGS. 8B and 8C.

### Example 11. Distribution of CAR-T Cells in a Tumor of the Female NSG Mice Bearing Human Colorectal Cancer Model as Measured by ddPCR

This example illustrates cellular kinetics of CAR-T cells within a tumor for thirty days post-administration of CAR-T cells. Each mouse received CAR-T positive cells at a dose of 1.0×106 cells.

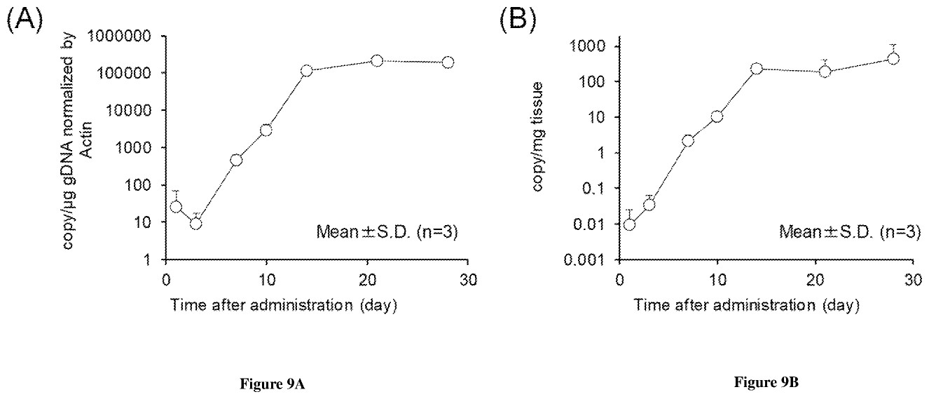

FIG. 9A depicts cellular kinetics of CAR-T cells within a tumor of female NSG mice expressed in terms of copy number per microgram of gDNA as measured by ddPCR. gDNA of CAR-T transgene was normalized using alpha actin, a single copy reference gene. FIG. 9B depicts cellular kinetics of CAR-T cells within a tumor of female NSG mice expressed in terms of copy number per milligram of tumor tissue as measured by ddPCR.

The cellular kinetics profile of CAR-T cell expressed in terms of copy number per microgram of gDNA was well correlated with that CAR-T cells number expressed in terms of copy number per milligram of tumor tissue. However, the transgene level expressed in copy number per microgram of gDNA, as shown in FIG. 9A, the CAR-T cell expansion as compared to the transgene level expression in terms of copy number per milligram of tumor tissue, as shown in FIG. 9B.

### Example 12. Comparison of gDNA Concentration Measurement by a Spectrophotometer and Reference Gene Measurement by a ddPCR

This example validates the reliability and robustness of gDNA measurement by ddPCR by comparing gDNA concentration measurement by ddPCR with the gDNA measurement by a spectrophotometer. Human gDNA concentration was measured by a spectrophotometer and a reference gene, alpha actin was measured by ddPCR. The spectrophotometer's absorbance value was converted into μg/mL of double stranded DNA (dsDNA) using the established conversion factor of 50 at 260 nm. The ddPCR-based reference gene copy number/μL was converted into μg/mL of gDNA using the formula described in FIG. 6B.

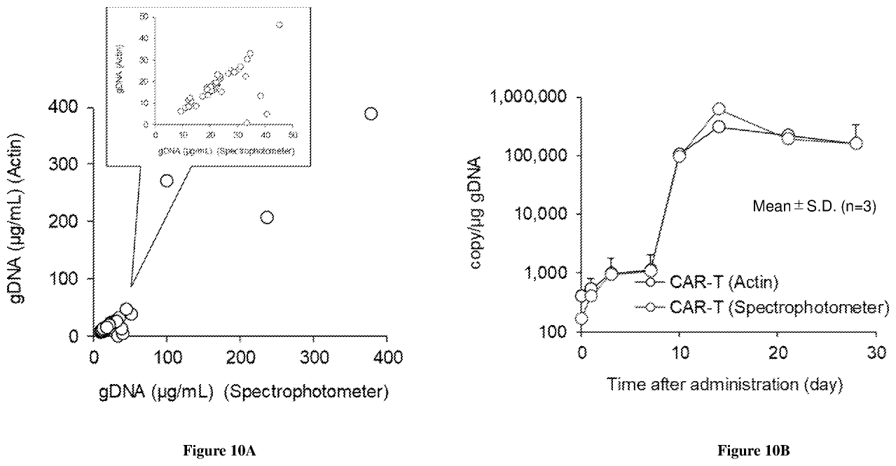

FIG. 10A depicts comparison of gDNA concentration measurement by a spectrophotometer and a reference gene detection by ddPCR. FIG. 10B depicts expansion of CAR-T cells over 30 days post-administration in terms of CAR-T transgene copy number per μg gDNA normalized by alpha actin as measured by a ddPCR (open black circle) and a spectrophotometer (open red circle).

As it is evident, gDNA concentration determined by a spectrophotometer and reference gene concentration measured by ddPCR, in general, matched quite well. However, the spectrophotometer measurement displayed a tendency to overestimate the copy number per microgram gDNA compared to that of measured by ddPCR (normalized by reference gene) likely due to any potential contamination of RNA or organic solvent residue in the eluate especially during early time point post-administration of CAR-T cells.

Sequence Table

| Target | | | SEQ ID |
|---|---|---|---|
| (gene) | Primer/probe | Sequence (5′-3′) | NO. |
| CAR-T | CD28F1 | CCCACCCGCAAGCATTACCA | 1 |
| | CD3ζR1 | GGTTCTGGCCCTGCTGGT | 2 |
| | CAR-T probe | (6-FAM)TCGCTCCAG(Zen)AGTG | 3 |
| | | AAGTTCAGCAGGAGC(IABKFQ) | |
| Alpha | Alpha actin_F1 | ATCGCTGACCGCATGCAGA | 4 |
| actin | Alpha actin_R1 | TCGTCGTACTCCTGCTTGGTG | 5 |
| | Alpha actin probe | (Hex)CCATCCTGG(Zen)CCTCGCTGTCCACC | 6 |
| | | (IABKFQ) | |
| HIS3 | HIS F1 | CGCAAATCCTGATCCAACC | 7 |
| | HIS R1 | ACTGAAGACTGCGGGATTG | 8 |
| | HIS probe | (6-FAM) | 9 |
| | | AGGGCCTCT(Zen)TTAAAAGCTTGACCGA(IABKFQ) | |
| LEU2 | LEU F1 | TGCTAAAGGTACTGACTTCGTTG | 10 |
| | LEU R1 | GATTCTTTGCACTTATGGAACG | 11 |
| | LEU2 probe | (Hex)ATGGTGATG(Zen) | 12 |
| | | GTGTCACTTGGGA (IABKFQ) | |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of determining a transgene copy number in a biological sample, the method comprising:
(a) extracting genomic DNA (gDNA) from the biological sample using an automated process, thus isolating gDNA;
(b) performing digital droplet PCR (ddPCR) on the isolated gDNA using one or more primers to amplify a transgene of interest and to amplify a reference gene, thus normalizing gDNA input; and
(c) determining transgene copy number in the biological sample.

2. The method of claim 1, wherein the transgene copy number in the biological sample is measured in copy number/μg gDNA

3. The method of claim 1, wherein the transgene copy number is determined using the following formula:
$$\frac{copy}{μg gDNA} = \frac{\frac{transgene copy}{μL reaction} \times 1,000,000}{\frac{reference gene copy}{μL reaction} \times \frac{3.3 pg}{copy}} .$$

4. The method of claim 2, wherein the copy number/μg gDNA unit is converted to copy number/μL blood by measuring gDNA extraction recovery.

5. The method of claim 4, wherein gDNA extraction recovery is assessed by spiking a known amount of a control gene before and after the gDNA extraction process.

6. (canceled)

7. The method of claim 5, wherein the gDNA extraction is stable as assessed by a quality control concentration of a control exogenous gene, and wherein the quality control concentration is selected from low quality control (LOC), medium quality control (MQC), or high quality control (HQC) concentration.

8. (canceled)

9. The method of claim 4, wherein gDNA extraction recovery is assessed by spiking a known amount of a chimeric antigen receptor (CAR) T cell before the gDNA extraction process.

10. A method of determining a transgene copy number in a biological sample, the method comprising:
(a) extracting genomic DNA (gDNA) from the biological sample using an automated process, thus isolating gDNA;
(b) determining a genomic DNA recovery rate by spiking in a known amount of a control gene before and after the gDNA extraction process;
(c) performing digital droplet PCR (ddPCR) on the isolated DNA using one or more primers to amplify a transgene of interest and to amplify the control gene, thus normalizing gDNA input;
(d) determining transgene copy number in the biological sample.

11. (canceled)

12. The method of claim 9, wherein the transgene copy number in the biological sample is measured in copy number/μL blood or copy number/mg tissue.

13. The method of claim 10, wherein the transgene copy number in the biological sample is measured in copy number/μL blood, using the following formula:
$$\frac{copy}{μ L blood} = \frac{transgene copy}{μ L of PCR reaction buffer} \times \frac{total μ L of PCR reaction buffer}{μ L of eluate used} \times \frac{total μ L of eluate}{total μ L of blood used} \times \frac{1}{genomic DNA recovery ( \% )} .$$

14. (canceled)

15. The method of claim 10, wherein the transgene copy number is in the biological sample is measured in copy number/mg tissue, using the following formula:
$$\frac{copy}{mg tissue} = \frac{transgene copy}{μ L of PCR reaction buffer} \times \frac{total μ L of PCR reaction buffer}{μ L of eluate used} \times \frac{total μ L of eluate}{total mg of tissue used} \times \frac{1}{genomic DNA recovery ( \% )} .$$

16. (canceled)

17. The method of claim 1, wherein the limit of quantitation (LOQ) is less than 50 copies of the transgene per 1 μg of gDNA.

18. The method of claim 1, wherein the reference gene is a single copy gene per haploid human genome and mouse genome.

19. The method of claim 15, wherein the reference gene is selected from alpha actin, RNase P gene, gamma tubulin 1, or GAPDH.

20. (canceled)

21. The method of claim 1, wherein the selectivity of ddPCR is assessed by performing a measurement of a no template control sample.

22. A method of determining cellular kinetics of administered CAR-T cells, the method comprising determining a CAR-T transgene copy number in accordance with claim 1.

23. A method of determining dose selection and dosing schedule for administration of CAR-T cells to a subject in need thereof, the method comprising determining CAR-T transgene copy number in a biological sample in accordance with claim 1, and adjusting CAR-T dose selection and/or dosing schedule to achieve a therapeutically effective amount of CAR-T cells in the subject.

24. A kit for determining a transgene copy number in a biological sample, comprising primers to amplify a transgene of interest and to amplify a reference gene, and instructions for determining the transgene copy number in the biological sample.

25. The kit of claim 24, wherein the instructions for determining the transgene copy number comprises the following formula:
$$\frac{copy}{μg gDNA} = \frac{\frac{transgene copy}{μL reaction} \times 1,000,000}{\frac{reference gene copy}{μL reaction} \times \frac{3.3 pg}{copy}} .$$

26. The kit of claim 24, further comprising one or more purified control genes, wherein the instructions for determining the transgene copy number comprises the following formula:
$$\frac{copy}{μ L blood} = \frac{transgene copy}{μ L of PCR reaction buffer} \times \frac{total μ L of PCR reaction buffer}{μ L of eluate used} \times \frac{total μ L of eluate}{total μ L of blood used} \times \frac{1}{genomic DNA recovery ( \% )} .$$

27. (canceled)
